(19) United States
(12) Patent Application Publication
(10) Pub. No.: US 2010/0179918 A1
(43) Pub. Date: Jul. 15, 2010

(54) PILOT ANTHROPOMETRIC SCREENING SYSTEM

(76) Inventor: Pierre Meunier, King City (CA)

Correspondence Address: BRION RAFFOUL, P O BOX 40060, 2515 BANK STREET, OTTAWA, ON, K1V 0W8, CA

(21) Appl. No.: 12/312,193

(22) PCT Filed: Nov. 1, 2007
(86) PCT No.: PCT/CA2007/001953

§ 371 Date: Mar. 4, 2010

Related U.S. Application Data
(60) Provisional application No. 60/856,046, filed on Nov. 2, 2006.

(51) Int. Cl. G06Q 10/00

(52) U.S. Cl. 705/348

(57) ABSTRACT

Methods and systems for determining a subject's suitability for workstations such as aircraft cockpits, based on the subject's anthropometric measurements. The subject's measurements are used as input to a workspace accommodation model that predicts an individual's ability to perform the tasks that are judged to be critical to the safe operation of said workspace. The workspace accommodation model is derived from experimentation and testing to determine the critical anthropometric measurements which will allow a subject to acceptably accomplish the tasks required for the workspace. Furthermore, the model is modular and adaptable such that it can improve the accuracy of its predictions with new cases, and thus learn over time.

### FIELD OF THE INVENTION

The present invention relates to human anthropometric measurements in relation to workstations such as aircraft cockpits. More specifically, the present invention relates to methods and systems for determining a subject's suitability in terms of their ability to safely perform all physical tasks related to flying an aircraft based on that subject's anthropometric measurements.

### BACKGROUND OF THE INVENTION

The appeal of flight and the temptation of piloting an aircraft is one that is familiar to most young people. Indeed, many a budding pilot has joined the military for a chance to control and pilot the various types of military aircraft available. Unfortunately, as many have found, enthusiasm and willingness to become a pilot is insufficient—the budding pilot actually has to fit in the cockpit of the aircraft. To this end, most military services have physical standards which determine who is to be admitted to flight school and who is to be eligible for training on which aircraft. These standards generally take into account only a few physical dimensions such as a subject's height, weight, sitting height, etc. without regard to whether he or she can safely operate a particular aircraft.

While such standards are used to screen applicants, they do not guarantee the suitability of an individual for flying a particular aircraft. In other words, a candidate may not be able to adequately perform all tasks attendant with being in a given cockpit. As was tragically found in an accident involving an American AH-64D gunship in 2006, a candidate who was screened into the training program through the use of a limited set of anthropometric criteria, was not able to adequately perform all of the critical tasks of that cockpit. In the accident, it was determined that the pilot's size was the main contributing factor as his body interfered with the range of motion of the stick and the pilot was therefore not able to manoeuvre the aircraft to its full potential. Because of the pilot's inability to fully manipulate some of the critical controls, the aircraft crashed, killing the pilot. (FL1GHTfax May 2006, Vol. 30, No. 5 pages 4-9).

Current anthropometric standards allowed the pilot to fly the aircraft simply because the anthropometric variables and their limits are not related to an individual's ability to perform all physical flight tasks.

To prevent future occurrences such as that noted above, improved methods and systems for determining a subject's suitability for a particular aircraft are required. Preferably, such methods and systems would automatically determine, for each subject, that subject's employability in the air force, or in other words their suitability for multiple aircraft.

### SUMMARY OF THE INVENTION

The present invention provides methods and systems for determining a subject's suitability for workstations such as aircraft cockpits, based on the subject's anthropometric measurements. The subject's measurements are entered into a workspace accommodation prediction model that determines not only if an individual is suitable for a particular workspace, but also provides the details of where he or she fails to achieve the required performance. The workspace accommodation model is derived from experimentation and testing to determine the critical anthropometric measurements as well as the way in which they combine to predict a subject's ability to accomplish the tasks required for the workspace. Only the tasks that are critical to the safe operation of the workspace are modeled.

In an aspect of the present invention, there is provided a method for determining a subject's physical suitability for a specific workspace in a specific aircraft, the method comprising a) receiving said subject's anthropometric measurements, b) retrieving a workspace accommodation prediction model for said aircraft, said workspace model containing detailed assessments of tasks that need to be performed in said workspace, c) using anthropometric measurements with corresponding models to determine if said subject meets the task-specific criteria, d) in the event said subject meets said task-specific criteria, determining that said subject is suitable for said specific workspace, e) in the event said subject does not meet at least one critical requirement, determining that said subject is unsuitable for said specific workspace.

In another aspect of the present invention, there is provided a mechanism by which said accommodation prediction model can improve its accuracy through the addition of data points or test cases. In this manner, the model has the ability to learn over time.

### BRIEF DESCRIPTION OF THE DRAWINGS

A better understanding of the invention will be obtained by considering the detailed description below, with reference to the following drawings in which:

FIG. 1 is a flowchart illustrating the steps in a method for screening subjects for suitability to particular workspaces in an aircraft according to one aspect of the invention;

FIG. 2 is a flowchart illustrating the steps for creating a workspace accommodation model to be used in the method of FIG. 1; and

FIG. 3 is a flowchart illustrating the steps for updating the workspace model used in the methods of FIGS. 1 and 2.

### DETAILED DESCRIPTION

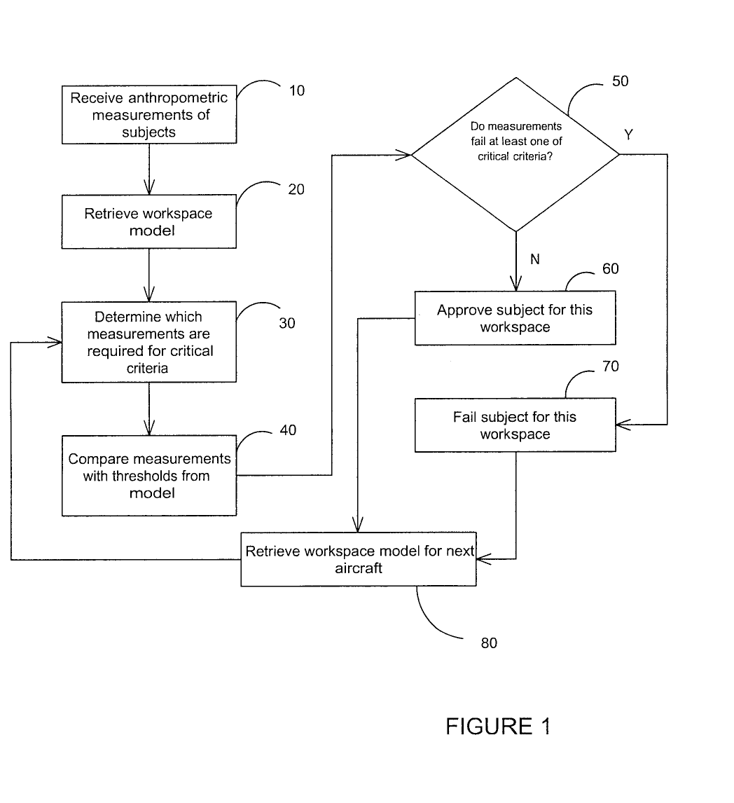

Referring to FIG. 1, a flowchart detailing the steps in an aircraft suitability method is illustrated. The method determines whether a subject (such as a candidate student pilot) is physically suitable for a workstation (such as a cockpit) in a particular aircraft. The method determines the subject's suitability based on whether the subject's physical dimensions will allow him or her to perform the tasks required by that workstation.

The first step (step 10) is that of receiving the subject's anthropometric measurements. These measurements are not limited to the subject's height, weight, but may include the subject's thigh circumference, eye height (when sitting), waist depth, sitting height, knee height, buttock to knee length, hip breadth, biacromial (distance between the shoulders) breadth, and any other measurements which may be relevant. The measurements may be gathered using any of the known methods, some of which may involve the use of lasers, imaging devices, and other modern devices.

The next step (step 20) is that of retrieving the workspace accommodation model for a particular aircraft against which the measurements received in step 10 will be applied. The workspace accommodation model contains the various task-specific models that will be used to determine whether a subject will be able to execute the various tasks associated with the workspace. The model not only details univariate limits of accommodation (e.g. maximum and minimum sitting height for a particular cockpit, maximum knee height so the knee will not touch an instrument panel, maximum weight which a seat can support, etc.) but also multivariate limits of workspace accommodation (e.g. combination of anthropometric variables and seat position, etc).

Once the model for the particular aircraft has been retrieved, the measurements related to the critical criteria are used to predict whether the subject is able to adequately perform critical tasks in the workspace. The critical criteria are therefore a subset of the workspace model. As an example, for a rotary wing aircraft, it might be determined that full cyclic stick authority is critical for the safe operation of the aircraft through its flight envelope. The model may take into account the seat position, thigh circumference and leg length as input variables to predict accommodation. As another example, to properly operate a specific type of aircraft, the subject must be able to reach a particular switch on a control panel (e.g. a battery switch, an auxiliary battery, or a general power switch). In this case, the model may take into account the seat position, acromial height sitting, and functional reach to predict reach performance. A further consideration may be whether the subject is able to fully actuate the controls (e.g. if the subject is able to exert enough force to press a button, to rotate a control yoke, to activate a switch, etc.) for such a consideration, the model may take into further account the subject's ability to actuate and/or manipulate the controls in addition to merely just being able to reach the controls.

The critical criteria may vary between aircraft as different aircraft have different layouts and hence different operational requirements for aircrew. Also, depending on the workstation, critical criteria may also differ between different workstations in the same aircraft. To simplify matters, the critical criteria for an aircraft may be stored as part of the workstation accommodation model for that aircraft.

As noted above, various different critical criteria may apply to different workspaces. The critical criteria may take into account a potential candidate's ability regarding vision (e.g. does the candidate have adequate vision of both the inside and outside of the cockpit) and reach/operability regarding instruments (e.g. can the candidate adequately reach and operate critical instruments such as switches, rudders, levers, etc.).

Step 40 then uses the relevant anthropometric measurements of the subject with the relevant models of the critical criteria determined in step 30.

Step 50 decides if the subject's anthropometric measurements meet all of the critical criteria. If at least one of the critical criteria thresholds is not met, then the subject is deemed to be physically unable to adequately perform at least one critical task or duty required for the workspace.

The assessment of the candidate's anthropometric measurements ideally takes into account various postures and seat positions. As an example, if a seat to be occupied by the candidate in an aircraft has 4 positions, each seat position would have its own critical criteria for various switches, rudders, and other instruments. These various critical criteria for each seat position must be assessed against the candidate's measurements. As an example, if seat position A is the closest to a specific critical panel, then the reach criteria for seat position A must be less onerous than for seat position B which would be further from the specific critical panel. If the candidate's reach meets or exceeds the criteria for position A but does not meet the criteria for position B, then the candidate would pass the critical criteria for position A but not for position B. As such, the candidate may be, depending on the configuration desired, listed as borderline for the overall critical criteria for reach. The testing for critical criteria for various seat positions also provides prospective pilots the safest seat positions to use when piloting their aircraft.

For a better indication of a subject's suitability or unsuitability based on seat position, a separate assessment may be made for each seat position and the results of this may be factored in the overall estimation of whether a subject passes or fails a particular task. In one implementation, for a subject to pass a critical criterion threshold that is affected by seat position, the subject's measurements must be given a pass for at least one seat position. The criticality of seat position may be made central to whether a subject obtains an overall pass or fail for an aircraft. Preferably, the subject must obtain a pass on all tasks for at least one seat position. This means that, for one specific seat position, all of the critical criteria thresholds must be passed in the subject's measurements. This would indicate that, for that one particular seat position, the subject is able to perform all tasks required to safely operate the aircraft.

As can be imagined, aspects of workspace accommodation is a multivariate problem, meaning that various variables are interconnected and interrelated. As an example and from the above, seat position can affect critical criteria thresholds—depending on the seat position, the critical criteria threshold can shift.

In the event the relevant criteria are met, the subject is approved (or cleared) for the particular aircraft for which the subject is being assessed (step 60).

If the subject's measurements do not meet the criteria it is assessed against, then step 70 indicates that the subject is not cleared for this particular workspace in this particular aircraft. However, a “pass” or “fail” for any criterion is preferably accompanied with a “confidence level” as to the passing or failing of the candidate for that criterion. As an example, if a verdict for a specific reach criterion is a “fail”, that verdict may have a confidence level of only 53%. Because of this comparatively low confidence level, this verdict may be tagged as “Borderline” and the candidate may be selected for a more thorough examination, perhaps even an actual in-cockpit testing. The confidence level may be generated based on database information generated from the general populace and specific in-cockpit or in-environment testing. Returning to the example above, the 53% confidence level may indicate that 53% of the people tested who had the candidate's reach measurements failed an actual in-cockpit reach test. However, because 47% of the people who had the same measurements passed the same reach test, then the candidate may be in the 47% category and, as such, more testing would be recommended.

In determining whether a subject passes or fails a particular task based on that subject's measurements, the methods for doing so may depend on the task itself. If the task has univariate limits, then this would simply mean that the subject's measurements are directly compared to a minimum or a maximum acceptable value (or a range of values) for that particular measurement and, based on whether the measurements meet or exceed the acceptable value, a pass or a fail is assigned. As an example, pilot seated height may be seen as a univariate issue. If a potential pilot is too tall to fit in a cockpit, then, clearly, the potential pilot would fail this critical criterion threshold. Similarly, multiple regression analysis may be used for multivariate limits—the results of multiple regression analysis of the subject's data can be compared to the require minimum or maximum or range of values and, based on this comparison, a pass or fail verdict can be generated. As is to be noted below, discriminant function analysis may be used for multivariate limits and the result of this would not only be a pass or fail verdict but also a confidence/probability level of the verdict based on the data in the database.

Once a verdict has been determined for one aircraft, the subject may be assessed against another aircraft's workspace model. To accomplish this, the new aircraft's workspace accommodation model is retrieved (step 80) and the critical criteria measurements are determined for this workspace model (step 30). The loop then continues as above. The method can continue to assess a subject's suitability for different aircraft and for different workspaces on the aircraft as long as workspace models are available.

In one implementation, the assessment of a subject may be done against models of a multiplicity of aircraft. This can determine which career path may be available to the subject based on the subject's anthropometric measurements. As an example, if trainer aircraft A is the only trainer aircraft for advanced aircrafts A1, A2, A3, and A4, then if the subject is unable to qualify/pass for aircraft A, then this means that the subject cannot pilot any of aircraft A1, A2, A3, or A4 as the subject cannot be trained on aircraft A. Similarly, if the subject is given a measurement pass for aircraft A, A2, and A4 but not for A1 or A3, then the subject can pilot aircraft A and, after training on that aircraft, the subject can move on to aircraft A2 and A4. As another example, if the subject obtains an overall pass on fixed wing aircraft A but not on rotary aircraft B and if B is the primary trainer aircraft for rotary aircraft, then that subject cannot pilot any of the rotary aircraft as he/she cannot be trained for rotary aircraft.

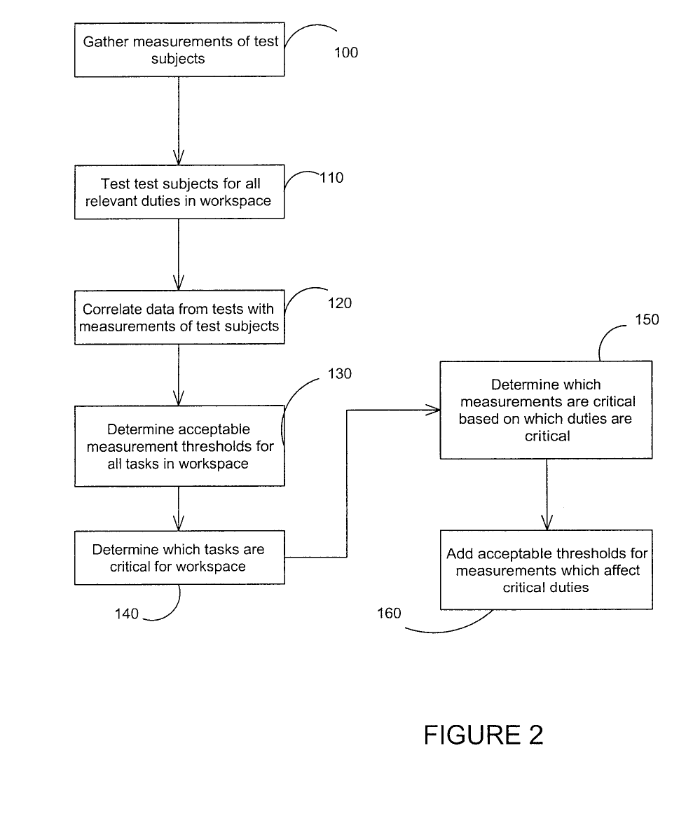

As can be seen from the above, the workspace model for a particular aircraft is a useful element of the method and decision system. In another aspect of the invention, the steps in workspace model creation are illustrated in FIG. 2.

As a first step in creating a workspace model for a particular aircraft, anthropometric measurements of test subjects are first gathered (step 100). These anthropometric measurements are, ideally, as extensive if not more extensive than those gathered for the subject discussed with relation to the method in FIG. 1. Gathering data from test subjects would provide the raw data for the database which would be used to determine the “pass” or “fail” confidence levels mentioned above.

The next step (step 110) is that of testing the test subjects in the aircraft and workspace to be assessed. This involves taking each test subject and simulating, as far as possible, the actual conditions under which the various tasks in the workspace are to be performed. This means that if possible, the test subjects are clothed in the actual uniforms to be used by the aircrew, complete with helmet, appropriate footwear, and gloves (if applicable). The test subjects are then asked to perform the various tasks associated with the specific workspace and their performance (or inability to perform) is noted. This may include reaching for specific switches, manipulating various control equipment such as yokes, joysticks, throttles, and the like. This may also include determining if the test subject can see outside the workspace and, if so, what is the test subject's range of vision. Further tests may include determining the amount of clearance between the subject and the aircraft structure and whether that clearance is sufficient for safe operation of the aircraft or safe ejection from it.

Step 120 is that of relating the test data gathered in step 110 with the measurements gathered in step 100. By relating the performance data with the measurement data through the use of multivariate statistical methods, a determination of which measurements affect the performance of which specific task can be made.

Once it has been determined which measurements affect the performance of which task, the univariate or multivariate thresholds for these measurements are determined (step 130). This involves determining what are the minimum acceptable measurement values for each task that ensures that the task can still be performed. Of course, in some cases, instead of a minimum, a maximum may be needed. In other cases, instead of a maximum or a minimum value, a range of acceptable measurement values are sought. As an example, for buttock to knee length, a maximum would be sought as a subject whose legs are too long would not fit in a specific cockpit. Similarly, a minimum arm reach length would be desired to ensure that the subject would be able to reach the instrument panels in the cockpit. For a range of acceptable values, sitting height would require such a range. A subject who is too short would be unable to see the landing aim point on final approach and a subject who is too tall would not fit in the cockpit.

It should be noted that discriminant function analysis methods may be used to generate the data used in the database. However, to adjust for the quirks of discriminant function analysis, a fair number of failures must be present in the data. This can be accomplished by ensuring that test subjects span a wide range of body sizes and shapes and by placing the test subjects in positions in which they are likely to fail. As an example, this would entail placing the test subject in all possible seat positions such that short test subjects would clearly not be able to perform the functions at the most extreme seat positions. As a further example, if a small subject is placed in a full-down and full-aft position, the short subject will fail the vision and overhead reach tests as well as possibly others. This would provide the failures required in the data by the discriminant function analysis method.

With the thresholds and ranges determined, a determination of which tasks and duties are critical to the workspace is made (step 140). This can be done by consultation with experts in the field or experience with the workspace and/or the aircraft. This is done to ensure that the criteria are indeed Bona Fide Operational Requirements (BFOR) and can be used as a basis for accepting or rejecting pilot candidates. The criticality of tasks and duties can be assessed by how important a task or duty is to the operation of the aircraft or to the safety of the crew. As an example, if an aircraft operator is required to see the landing aim point on a flapless final approach, then a minimum acceptable vision over the nose needs to be specified. As another clear example, the ability to fully manipulate the control stick, the yoke, throttle, and other control implements is important to the safe operation of the aircraft.

Once a listing of the critical tasks related to the workspace has been compiled (again through consultation with operators of or experts on the specific aircraft), this list is correlated with the results of step 120 to determine which anthropometric measurements are critical to the workspace in the aircraft (step 150). This step isolates the anthropometric measurements that are critical to the proper operation of the aircraft or for the proper performance of the tasks associated with the workspace. If one of the performance criteria associated with these anthropometric measurements is not met, then it would be predictable that the subject with the anthropometric measurement values that do not meet one of the thresholds would be unable to adequately perform a critical task associated with the workspace. In this sense, by relating adequate performance in the workspace with anthropometric measurements of subjects, it is possible to predict the performance of any given subject in that workspace.

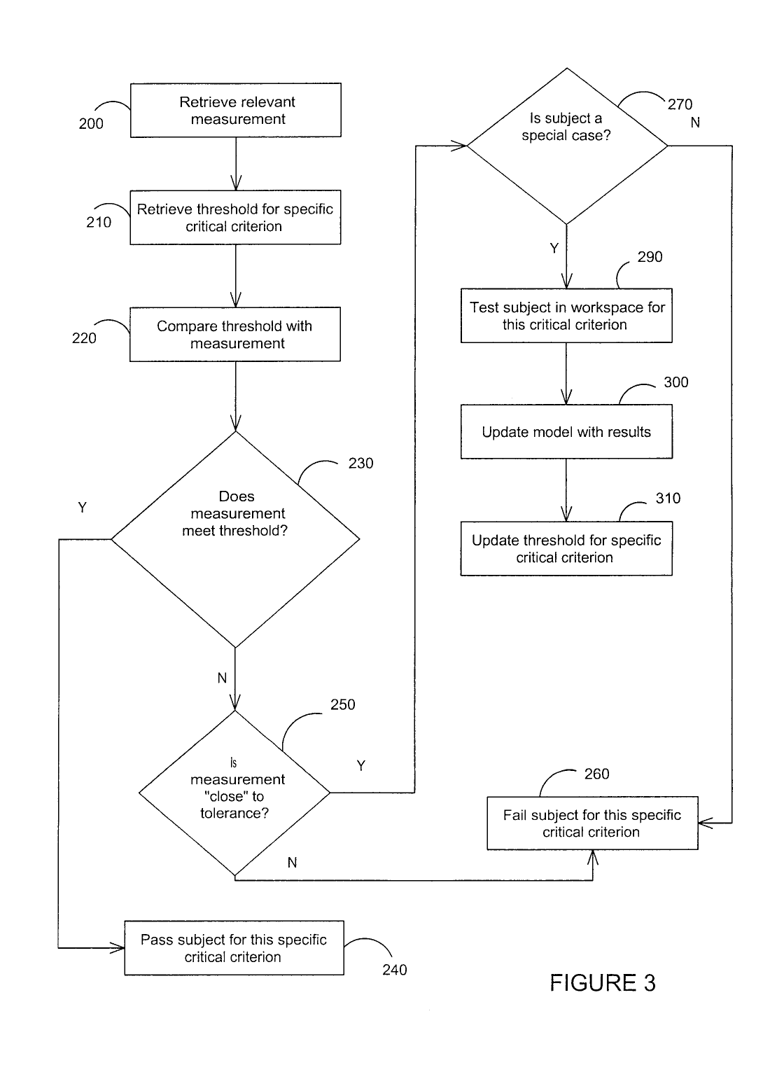

It should be noted that the workspace accommodation models may be updated as needed as new workspaces are added. However, the workspace model can be constantly updated for existing workspaces as new data points are received as a result of in-cockpit testing of borderline candidates. This is done by executing the method illustrated in the steps outlined in the flowchart of FIG. 3 and is provided in the context of testing a particular subject for his or her suitability for a particular workstation. In this manner, the additional data serves to improve the model's accuracy as it “learns” from new cases.

The first step (step 200) is that of receiving the relevant anthropometric measurements for the subject. These measurements relate to the specific critical criteria mentioned above that would determine the subject's suitability.

Step 210 retrieves the models (and the identification of the measurements associated with those models) for the critical criterion. As an example, if the critical criterion related to being able to see the landing aim point, the measurements associated with this could include the subject's eye height sitting, and seat position.

Once the models and the subject's relevant measurements have been retrieved, these are compared in step 220. A decision (step 230) then determines if the relevant measurements meet the relevant criteria (step 230). If the criteria are met, then the subject is cleared for that particular aircraft (step 240). On the other hand, if the subject's measurements do not meet the criteria, then another decision (step 250) is taken.

Depending on the results obtained, the verdict (step 250) may be that the candidate either passes or fails to meet the criteria, or that there is too much uncertainty in the model to decide one way or the other. That third possible verdict, “close”, represents a point where the probability or success or failure are similar and may require in-cockpit testing. Of course, if the probability of failure is greater than the predetermined threshold of acceptability, the subject is not cleared for this workspace (step 260).

If, on the other hand, the difference is within the predetermined tolerance limit, then decision 270 is made. Decision 270 determines if the subject is a special case for whom special consideration is warranted. If no special consideration is warranted, then the subject is rejected for this particular workstation (step 280).

If special consideration is warranted, then this particular subject is tested in the workspace to determine if he or she can execute the tasks associated with the measurements. Regardless of whether the subject can perform the tasks even with the non-adherence to the threshold, this datum is added to the workspace accommodation model. If the subject can perform the tasks, then he/she is given an overall “pass” but, of course, if the subject is unable to perform a critical task, then he/she is given an overall “fail”. It should be noted that, preferably, the data for every subject tested is added to the database.

The model is updated in step 300 and this may involve changing the threshold for the specific critical criterion being tested (step 310).

It should be noted that steps 200-240 may be seen as a portion of a regular execution of the method in FIG. 1.

It should further be noted that while the above description discusses the invention with respect to aircraft workstations such as cockpits, the invention is equally applicable to any workstation that involves constrained physical space (with or without the added constraint of bulky equipment and/or uniforms) and critical tasks affected by anthropometric considerations. The invention is thus applicable to the assessment of candidates for workstations in vehicles or environments such as armored vehicles for the military, emergency response vehicles (e.g. emergency medical transport, ambulances, hazardous materials response vehicles, fire trucks, etc.) and others where physical size and physical measurements may affect a subject's performance.

Embodiments of the invention may be implemented in any conventional computer programming language. For example, preferred embodiments may be implemented in a procedural programming language (e.g. “C”) or an object oriented language (e.g. “C++”). Alternative embodiments of the invention may be implemented as pre-programmed hardware elements, other related components, or as a combination of hardware and software components.

Embodiments can be implemented as a computer program product for use with a computer system. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or electrical communications lines) or a medium implemented with wireless techniques (e.g., microwave, infrared or other transmission techniques). The series of computer instructions embodies all or part of the functionality previously described herein. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e,g., shrink wrapped software), preloaded with a computer system (e.g., on system ROM or fixed disk), or distributed from a server over the network (e.g., the Internet or World Wide Web). Of course, some embodiments of the invention may be implemented as a combination of both software (e.g., a computer program product) and hardware. Still other embodiments of the invention may be implemented as entirely hardware, or entirely software (e.g., a computer program product).

A person understanding this invention may now conceive of alternative structures and embodiments or variations of the above all of which are intended to fall within the scope of the invention as defined in the claims that follow.

What is claimed is:

1. A method for determining a subject's physical suitability for a specific workspace in a specific aircraft, the method comprising:
a) receiving said subject's anthropometric measurements
b) retrieving a workspace accommodation model for said aircraft, said workspace accommodation model using said anthropometric measurements to predict a person's ability to execute tasks for said specific workspace
c) using anthropometric measurements with corresponding criteria in said workspace accommodation model to determine if said anthropometric measurements of said subject meet said corresponding criteria, said corresponding criteria being related to measurements which a affect a person's ability to execute said tasks
d) in the event said anthropometric measurements or said subject meet said corresponding criteria, determining that said subject is suitable for said specific workspace
e) in the event said anthropometric measurements of said subject does not meet at least one critical criterion of said model, determining that said subject is unsuitable for said specific workspace.

2. A method according to claim 1 wherein said workspace accommodation model includes univariate and multivariate thresholds for physical measurements which will allow a person to function in said specific workspace.

3. A method according to claim 1 wherein step c) comprises determining if said anthropometric measurements exceed corresponding thresholds which relate to said criteria.

4. A method according to claim 1 wherein step c) comprises determining if said anthropometric measurements are less than corresponding thresholds which relate to said criteria.

5. A method according to claim 1 wherein at least one of said corresponding criteria comprises at least one threshold for physical measurements which, if not met, will prevent said person from executing a critical task for said specific workspace.

6. A method according to claim 1 wherein step c) is only executed for critical thresholds which correspond to said corresponding criteria.

7. A method according to claim 1 wherein said workspace accommodation model is derived from test data obtained by measuring performance of test subjects when performing tasks in said specific workspace.

8. A method according to claim 7 wherein said model is derived from relating physical measurements of test subjects with said test subjects' ability to perform said tasks in said workspace.

9. A method according to claim 8 wherein said tasks are critical tasks.

10. A method for determining a subject's physical suitability for a specific workspace, the method comprising:
a) receiving said subject's anthropometric measurements;
b) retrieving a workspace accommodation model, said workspace accommodation model containing thresholds for physical measurements which will allow a person to execute tasks for said specific workspace;
c) using anthropometric measurements with corresponding models to determine if said anthropometric measurements of said subject meet said corresponding thresholds;
d) in the event said anthropometric measurements of said subject meet said corresponding thresholds, determining that said subject is suitable for said specific workspace;
e) in the event said anthropometric measurements of said subject does not meet at least one critical threshold of said thresholds, determining that said subject is unsuitable for said specific workspace.

11. A method according to claim 10 wherein said thresholds comprise univariate or multivariate thresholds.

12. A method according to claim 1 wherein said workspace accommodation model includes univariate and/or multivariate thresholds for physical measurements which will allow a person to manipulate and/or actuate controls in said specific workspace.

13. A method for determining a subject's physical suitability for a specific workspace the method comprising:
a) receiving said subject's anthropometric measurements
b) retrieving a workspace accommodation model for said workspace, said workspace accommodation model having predetermined criteria for anthropometric measurements for determining a person's ability to execute specific tasks for said specific workspace
c) using said anthropometric measurements received in step a) with corresponding criteria in said workspace accommodation model to determine if said anthropometric measurements of said subject meet said corresponding criteria, said corresponding criteria being related to measurements which a affect a person's ability to execute said tasks
d) in the event said anthropometric measurements or said subject meet said corresponding criteria, determining that said subject is suitable for said specific workspace
e) in the event said anthropometric measurements of said subject does not meet at least one critical criterion of said model, determining that said subject is unsuitable for said specific workspace
f) adding said anthropometric measurements of said subject and results of step c) to a database of measurements for use in determining a future subject's suitability for said specific workspace.

14. A method according to claim 13 wherein step c) further comprises generating a pass or fail verdict for whether said subject's anthropometric measurements meet said corresponding criteria.

15. A method according to claim 14 wherein step c) further comprises generating a confidence level regarding said verdict based on data from said database.

16. A method according to claim 14 wherein step c) comprises using discriminant function analysis to determine said verdict.

17. A method according to claim 13 wherein said workspace accommodation model contains varied predetermined criteria for more than one seat position in said specific workspace.

18. A method according to claim 17 wherein, to determine if said subject is suitable for said specific workspace, said anthropometric measurements of said subject meet all corresponding criteria for critical tasks in said specific workspace for at least one seat position.

19. A method according to claim 13 wherein data in said database is generated by testing a variety of subjects within said specific workspace for whether said variety of subjects can accomplish specific tasks in said specific workspace.

20. A method according to claim 13 wherein said anthropometric measurements include at least one of the following: height, weight, thigh circumference, eye height, waist depth, sitting height, knee height, buttock to knee length, hip breadth, biacromial breadth.

21. A method according to claim 13 wherein said specific tasks relate to tasks critical to a safety of said workspace.

22. A method according to claim 13 wherein said specific tasks relate to tasks critical to a proper functioning of an environment of said specific workspace.
